(19) United States
(12) Patent Application Publication
(10) Pub. No.: US 2006/0029670 A1
(43) Pub. Date: Feb. 9, 2006

(54) Pharmaceutical Formulations and Method for Making

(75) Inventors: Torsten Hoffmann, Radebeul (DE); Michael Pieroth, Weinbohla (DE); Gerhard Zessin, Halle/Saale (DE); Karl-Friedrich Landgraf, Dresden (DE)

Correspondence Address: GUDRUN E. HUCKETT DRAUDT, LONSSTR. 53, WUPPERTAL, 42289, DE

(73) Assignee: AWD.PHARMA GMBH + CO. KG, Dresden (DE)

(21) Appl. No.: 11/163,297

(22) Filed: Oct. 13, 2005

Related U.S. Application Data
(62) Division of application No. 09/793,936, filed on Feb. 27, 2001.
(60) Provisional application No. 60/187,962, filed on Mar. 9, 2000.

(51) Int. Cl. A61K 31/7024; A61K 9/26

(52) U.S. Cl. 424/469; 514/53

(57) ABSTRACT

The invention relates to a process of making an oral pharmaceutical formulation with variably adjustable release rate, which comprises one or more active ingredients, and one or more sucrose ester of a fatty acid as the sole release-controlling agent for said active ingredient wherein when the dosage form is a granule or a pellet. The formulation is made by melting the oral formulation and granulating or pelletizing the melt.

This application is a divisional of co-pending U.S. application Ser. No. 09/793,936, filed Feb. 27, 2001, which claims the benefit of priority of U.S. provisional patent application No. 60/187,962, filed Mar. 9, 2000; both of which are hereby expressly incorporated by reference in their entireties.

### BACKGROUND OF THE INVENTION

The present invention relates to new oral pharmaceutical formulations with variably adjustable release characteristics for the active ingredient, suitably in the form of granulates, pellets, tablets, film coated tablets, microtablets, sugar coated tablets, capsules or therapeutic systems, as well as to methods for their manufacture by melt granulation or melt pelletization.

A reduced frequency of taking medicinal drugs and, in the ideal case, taking such drugs only once daily can play an important role in their use. One tablet in the mornings or the evenings is usually taken more regularly than are several tablets spread over the day. In addition to the convenience, this improved patient compliance also has a positive effect on the healing process. In addition, the better compatibility of the active ingredient, which is frequently associated with a reduced frequency of taking it, benefits the patient. The latter is related to the need to maintain the effective plasma concentration for a longer time and to the mostly more uniform plasma levels, at which incompatible peak levels are largely avoided.

In exceptional cases, a single administration can already be realized by the kinetic or dynamic properties of an active ingredient, such as by a long elimination half-life. In most cases, however, effective plasma levels over 12 to 24 hours become possible only by pharmaceutical and technological measures, such as the delayed release of the active ingredient from the form in which it is administered.

The literature describes a series of solutions which in principle, depending on the chemical and physical properties of the active ingredient, have advantages or disadvantages (e.g. see the review article: Recent Trends and Progress in Sustained or Controlled Oral Delivery of Some Water Soluble Drugs, Drug Development and Industrial Pharmacy 21 (9), 1037-1070 (1998)).

The state of the art is given, for example, also in one of the newer textbooks of pharmaceutical technology (Voigt, R., Pharmazeutische Technologie (Pharmaceutical Technology), Ullstein Mosby Publishers 1993, page 293 ff.). According to this, the action of drugs can be prolonged by measures such as: varying the molecule, for example, by forming a salt or an ester, changing the active ingredient modification, the particle size, the choice of appropriate inert ingredients and the appropriate methods. Some exemplary possibilities are discussed below.

(a) Matrix Forms for Controlled Release of Drugs

These are characterized by an insoluble, possibly porous framework of indigestible fats, waxes, polymers or also inorganic matrix-forming materials. The active ingredient is incorporated into this framework and released by diffusion, erosion or matrix decomposition.

(b) Hydrocolloid Forms for Controlled Release of Drugs

The drug is incorporated in this case hydrocolloid matrices, such as cellulose derivatives. After the drug is ingested, a gel is formed by the digestion fluids. The active ingredient diffuses from the gel at a rate, which depends on the surface area and the gel viscosity.

(c) Coated (Membrane-Controlled) Forms for Controlled Release of Drugs

Active ingredient particles or drug forms are enveloped in these cases by a barrier. Diffusion through the diffusion barrier determines the rate of release of the active ingredient. Plasticizers or pore-forming agents can be added to increase the diffusion rate.

(d) Effect of the Specific Surface Area

For active ingredients having a low water solubility, there is generally a clear relationship between their rate of dissolution and their specific surface area. A defined particle size distribution and, and thus a particular specific surface area can be achieved by selective crystallization of the active ingredient, by screening or by grinding. The larger the particles, the smaller is the specific surface area and the slower is the release of active ingredient.

(e) Mixed Forms of Diffusion, Erosion and Dissolving Processes

Drug forms are known, for which the delayed release of the active ingredient is based on a combination of diffusion, erosion and dissolving processes.

Melt granulation represents a particularly interesting and, with respect to the release of active ingredient, very variably usable method. Melt granulation or thermoplastic granulation is a process, for which granulate bonding is brought about through the use of a low-melting component, as well as under the influence of thermal energy (Lüdemann, J.: APV Course 231 of Jun. 17 to 18, 1996).

A differentiation is made here between two sub-types. In the case of wet granulation, the process temperature is above the melting point of the binding component. The latter is present during the granulation as a liquid or semi-solid component. In melt granulation, drying is replaced by cooling.

Melt granulation is a sinter granulation, when the process temperature does not reach the melting point of the binding component. Only local melting at the surface of the particles takes place, so that the surfaces diffuse into one another (Voigt, R: Lehrbuch of pharmezeutischen Technologie (Textbook of Pharmaceutical Technology), Verlag Chemie, page 159 (1984)).

The low-melting component can be an active component or an inactive ingredient. For stability reasons, the melting points of the substances are generally above 35° C. The most frequently used materials have melting points ranging from 500 to 90° C. Known active ingredients, as fusible substances, are phenyl salicylate, ibuprofen, a-liponic acid and meprobamate. Water soluble, swellable and lipophilic substances are used as fusible inert ingredients. For example, Macrogol, Polyvidon and polymethacrylic acid derivatives are used as hydrophilic materials. Hydrocarbons (paraffins), waxes, fats and fatty acids are examples of inert lipophilic materials. (Flanders, P.; Dyer, G. A.; Jordan, D.; Drug Dev. Ind. Pharm. 13 (&), 1001-1022 (1987); Schaefer, T.; Holm, P.; Kristensen, H. G.; Drug Dev. Ind. Pharm. 16, 1249-1277 (1990); McTaggart, C. M. et al.; Int. J. Pharm. 19, 139-148 (1984); Kinget, R.; Kernel, R.; Acta Pharm. Technol. 31, 57 (1985)).

Melt granulation is usually carried out in fluidized bed granulators, centrifugal fluidized bed equipment or high-speed intensive mixers. The use especially of the latter has processing advantages, since a cost intensive air preparation can be omitted. Compared to conventional granulation methods with organic solvents. There are no expenses for explosion protection and solvent recovery compared to nonaqueous granulation. There are also no residual solvents in the product. There are no energy-consuming drying processes. The use of so-called one-reactor systems is preferred in such cases.

The process of melt granulation can in general be shown as follows:

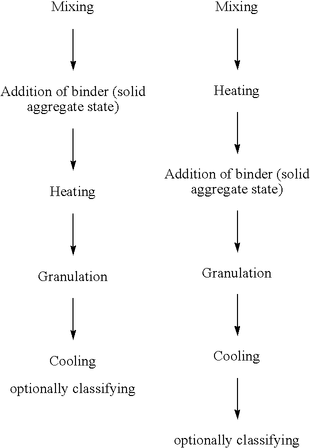

Fusible binders can be added in the solid or liquid state, that is, in the molten state.

For solid addition, the fusible material is melted during the process. For this reason, this method is also referred to as the melting method.

For the latter method, either the solid components are transferred to the reactor first and the liquid binder is added or, corresponding to the so-called fusion method, the liquid binder is added to the reactor and the solid materials are stirred in. For this purpose, heating is carried out before the addition of the binder.

In the case of intensive mixers, energy can be supplied in various ways:
mechanical energy by mixing tools and choppers;
contact heat by way of a heating jacket;
radiation energy by IR or microwave;
hot air introduction into the product bed.

A large number of methods for producing such formulations are also known from the patent literature. Formulations with a controlled release, which can be produced by way of melt granulation, are described, for example, in German patent No. 2,426,812, European patents 351,580; 654,263; 672,416; and 729,751 and in WO 93/18753. The last one describes a process, in which water-insoluble, hydrophobic, wax-like substances are added at a later time in the production process to the prepared pellets at a temperature, at which these substances melt and lead to a coating of these substances. This process is referred to as “hot-melt coating”.

On the assumption that all of the starting materials, participating in the process are thermally stable under the existing process conditions, melt granulation is an interesting alternative to other granulation methods, such as granulating, for example, with organic solvents or granulating with water.

Melt pelletizing represents a special form of carrying out the process, for which the granulate particles are produced with a largely uniform size and rounded shape.

In spite of the large number of known non-active excipients, which can be melted, only a few such materials with graded HLB values (hydrophilic-lipophilic balance values) are described, which are particularly suitable for melt granulation processes or melt pelletization processes.

Representatives of the few inert ingredients with graded HLB values are hydrogenated edible fats, which are available under the trade name of Gelucires, or the sorbitol esters of fatty acids, which are known for example, as Span. However, these also do not cover the broad HLB range from 1 to 16.

With the classical, fusible inert ingredients, the release rates can be varied only by the retarding agent selected or by the amount of this agent. Frequently, a binder can be processed only in combination with a different fusible binder, such as polyethylene glycol, since its granulate-forming alone is inadequate. These binders also require the addition of lubricants or mold release agents. Some have a wax-like consistency. In the case of the known methods of melt granulation, the resulting, solidified granulates must frequently be subjected to an expensive screening process to comminute the product.

When preparing controlled release compositions by a coating procedure, destruction of the film coating is frequently observed during pressing because of the partially brittle, but also relatively thin film coatings, unless such a destruction is counteracted with a relatively large amount of external phase. When the film coating is destroyed, the release rate of active ingredient from the tablets is increased. This means that the release of active ingredient from these tablets mostly depends on the pressing force. Frequently, in the case of this method, the release of active ingredient is adjusted by the amount sprayed on during the manufacturing process. Depending on the film formation and the porosity, the release rate of active ingredient may change during storage, for example, due to post-curing.

### SUMMARY OF THE INVENTION

The objective of the present invention to provide methods for producing oral pharmaceutical formulations with a variable, adjustable release behavior, which can range from rapid to retarded release. Through the present invention, it is possible to produce non-disintegrating drug forms (so-called “single units”) as well as suitable rapidly disintegrating and modified or retarded drug forms (so-called “multiple unit forms”) from the granulates, wherein the release rate of the active ingredient of the dosage forms are modified or retarded. The process of the present invention to for producing such retard or slow release formulations is achieved especially through melt granulation or melt pelletization.

According to the present invention, new oral pharmaceutical formulations with variably adjustable release behavior are provided which, in addition to one or more active ingredients, contain one or more sucrose esters of fatty acids as the sole release-controlling agent. The new pharmaceutical formulations are dosage forms, which release at various rates from immediate to controlled release.

As used throughout the disclosure and the claims, any reference to any active ingredient is meant also to include optionally more than one active ingredient, and reference to a sucrose ester of a fatty acid also includes optionally more than one sucrose ester of a fatty acid.

The pharmaceutical formulations of the present invention can be administered in the form of granules, pellets, tablets, film-coated tablets, microtablets, sugar-coated tablets and capsules and as therapeutic systems.

Surprisingly, sucrose esters of fatty acids are able to control the release of active ingredients in the desired manner and, moreover, to improve the technological properties during the preparation of the formulations of the invention by melt granulation or melt pelletization.

Sucrose esters of fatty acids are also suitable for granulating the active ingredient without the addition of other inert materials. By these means, a gross reduction in weight is possible in comparison to other methods, in which several fusible retarding agents or binders have to be used. At the same time, sucrose esters of fatty acids, particularly stearates with a low HLB value, such as from about 1 to about 16 can be suitably used as lubricants and as mold release agents.

Sucrose esters of fatty acids are nonionic surfactants, which are mono-, di-, tri- and polyesters of sucrose as the hydrophilic component and saturated or unsaturated fatty acids as the lipophilic component. By varying the degree of esterification and the nature of the fatty acids, sucrose esters of fatty acids can be produced with different HLB values, which have an effect on the biopharmaceutical properties, especially the release of active ingredient, the stability of the pharmaceutical formulation produced and its technological behavior. They are nontoxic, biodegradable, tasteless and odorless and have a long shelf life. The sucrose esters of fatty acids with a melting point higher than 30° C. are solid at room temperature and have an HLB value of from 1 to 16.

Sucrose esters of fatty acids are also sold under the name, for example, of sugar esters or sucrose esters by Mitsubishi (under the trade name of Ryoto), Gattefosse, or Sisterna and others.

Sucrose esters of fatty acids known from the literature are, for example, those of U.S. Pat. No. 4,844,067 used to improve the surface of silk fibers, and those of WO 93/17667 as taste improvers in pharmaceutical preparations.

Their main use is in the food industry. For example, sucrose esters of fatty acids are used to improve the mixing of chewing gum compositions, to counteract demixing and denaturing of finished beverages, for refining sugar, in condensed milk and in coffee creamers.

Sucrose esters of fatty acids are used for the production of wheat flour products, for example, as stabilizers to improve the texture and to avoid baking on and sticking on. In milk products they are used to stabilize emulsions and to avoid proteins and degradation, sucrose esters of fatty acids improve the crystallization behavior, and are effective emulsifiers and lower the viscosity during the production all fats and oils.

In U.S. Pat. Nos. 3,896,238; 4,150,114; and 4,046,886; the use of sucrose esters of fatty acids in combination with alkyl sulfoxide or phosphorus oxides in pharmaceutical compositions is disclosed for improving the penetration of the active substance through the skin. Sucrose monooctonate, monolaurate, monopalmitate and monostearate, as well as diesters and triesters of these compounds are named as special sucrose esters of fatty acids. In Japanese patent No. 8,175,437, the use of sucrose esters of fatty acids with an HLB value of 1 to 5 is disclosed as a base for suppositories.

In WO 88/06880, the use of sucrose esters of fatty acids in topical applications is disclosed, mixtures of mono- and dialkyl sucrose esters with an HLB value of 8 to 16 being used to improve the penetration through the skin. Preferably, sucrose cocoate, sucrose ricinoleate, sucrose laurate and sucrose stearate are used for that purpose.

Sucrose esters of fatty acids are also used, particularly, in cosmetic products (French patent No. 2,421,605, and Japanese patents Nos. 8,124,034 and 8,155, 306).

In German patent No. 4,003,844, pharmaceutical compositions are described which, in addition to the active ingredient, cyclosporin, contain a sucrose monoester of a fatty acid and a diluent or carrier. These compositions enable the cyclosporin dosage level, required for achieving an effective therapy, to be reduced and, thus, lead to a reduction in undesirable side effects. As sucrose monoester of a fatty acid, monoesters of C₆₋₁₄ and C₈₋₁₈ fatty acids are particularly suitable for that purpose.

In WO 93/00093, a new controlled release formulation for Diltiazem in the form of spheroids is disclosed, which is composed of the active ingredient, a wetting agent and a polymer coating for controlling the release. Sucrose esters of fatty acids are used as a wetting agent. The actual retardation of active release takes place by a polymer. Moreover, the wetting agent is processed with the active ingredients by extrusion or by granulation with organic solvents. The extrudates are coated with conventional polymers. Sucrose or xylose esters of C₁₂₋₂₀ fatty acids, for example, are named as wetting agents.

In German patent No. 19,840,152, a retard formulation is disclosed, which contains calcium valproate, at least one acrylic polymer and at least one sugar ester, wherein the desired retarding effect being achieved by the acrylic polymer that is used. It is shown that the sugar ester, by itself, does not have any meaningful release retarding effect.

The suitability of sucrose esters of fatty acids to be the sole release-controlling agent in the pharmaceutical formulations of the present invention was all the more surprising, since these sucrose esters of fatty acids, on the one hand, have already been known for a long time per se, and now in accordance with the present invention can be employed in a simple manner in oral pharmaceutical formulations, with a variably adjustable release behavior.

The sucrose esters of fatty acids, used pursuant to the present invention, are esters of sucrose with saturated or unsaturated fatty acids or mixtures thereof. Particularly suitable are C₁₂₋₂₂ fatty acids. Sucrose stearates, sucrose palmitates, sucrose laurates, sucrose behenates and sucrose oleates, with an HLB value of about 1 to about 16, are suitably used. The melting point or melting range of the sucrose esters of fatty acids, which are used pursuant to the invention, lies between about 30° C. and about 200° C. Suitably, sucrose esters of fatty acids with a melting point or melting range of from about 40° C. to about 150° C. are used.

An essential advantage of the present invention is that the desired release behavior of the new pharmaceutical formulations can be controlled by the type and proportion of the sucrose fatty acid ester or esters used and by the parameters of the manufacturing process. Selection of an appropriate ester or combination of esters, and suitable processing parameters can be determined as the basis of guidelines disclosed herein and by routine experimentation.

Sucrose esters of fatty acids with a low HLB value are suitable for achieving a retarded release. Sucrose esters of fatty acids with a high HLB value are suitable for a more rapid or modified release behavior.

In the pharmaceutical formulations of the present invention, the sucrose esters of fatty acids can be used in amounts of from about 1% to about 95% by weight, based on the amount to be granulated (inner phase) in the formulation. More suitably, an amount of about 5% to about 50% by weight is used. Aside from sucrose esters of fatty acids, the active ingredient or mixtures of the active ingredient can also contain one or more inert excipients, such as are conventionally used in pharmaceutical preparations in the inner phase.

In further embodiments of the invention granules or pellets, which may or not contain sucrose esters of fatty acids in the granulate, can be coated instead with sucrose esters of fatty acids. The proportion of sucrose esters of fatty acids in the coating is from about 1% to about 60% by weight and suitably from about 3% to about 20% by weight, based on the coated form of the drug.

The sucrose esters of fatty acids can be used by themselves or optionally also in combination with other fusible inert ingredients. In some cases, the addition of one or more inert materials, such as plasticizers, can be of advantage for the process. A further modification of the release of active ingredient is possible by way of embedding suitably during the melt granulating or melt pelletizing process, a so-called pore-forming agent, an inert material with certain properties, such as having a characteristic solubility or swellability.

As active ingredients, the inventive, oral pharmaceutical formulations can contain compounds, the solubility of which in water ranges from good to practically insoluble.

For example, active ingredients of the following indication groups were found to be suitable for this purpose, analeptic agents, antihypoxemic agents (such as caffeine), analgesics, antirheumatic agents (such as diclofenac, morphine, tramadol, tilidin, flupirtin), antiallergic agents (such as azelastin, pseudoephedrine), antiarrhythmic agents (such as quinidine, disopyramide, diltiazem, verapamil), antidementia agents (nootropic agents) (such as piracetam, nicergolin, xantino nicotinate, pentifyllin, vincamin), antidiabetic agents (such as glibenclamide), antiemetic agents, antivertiginous agents (such as betahistin dimesilate, dimenhydrinate), antiepilieptic agents (such as carbamazepine, valproic acid, calcium valproate dehydrate, retigabine), antihypertensive agents (such as talinolol, fosinopril, doxazosin, metoprolol, nifedipine), antihypotensive agents (such as norfenefrin-HC1, dihydroergotamine mesilate), broncholytic agents, antiasthmatic agents (such as salbutamol, terbutalin sulfate, theophyllin), diuretics (such as furosemide, piretamide), circulation promoters (such as buflomedil, naftidrofuryl, pentoxifyllin), hypnotic agents, cardiac agents (such as trinitroglycerin, isosorbid mononitrate, isosorbid dinitrate, molsidomin), sedatives, lipid-lowering agents (such as bezafibrate, fenofibrate, xantinol), antimigraine preparations (such as sumatriptan), muscle relaxants, anti-Parkinson agents and other agents against extrapyramidal disorders (such as levodopa, benserazide, carbi-dopa), psycho-pharmaceuticals (such as amitriptylin-HC1, venlafaxin-HC1, thioridazin-HC1, lithium carbonate, lithium acetate), or their pharmaceutically acceptable salts.

The pharmaceutical formulations of the present invention can suitably contain flupirtin, tramadol, nifedipine, carbamazepine, calcium valproate or retigabine.

Pursuant to the present invention, the pharmaceutical formulations of the invention can be suitably prepared by melt granulation or melt pelletization. For this purpose, for example, the mixture of active ingredient and one or more sucrose esters of fatty acids is heated with stirring in a high-speed mixer, optionally together with other inert materials. The heating can be accomplished by a heating jacket, with microwave, by radiation energy or by supplying energy by stirring. Granulation commences when the melting temperature of the sucrose ester of the fatty acids used in the mixture is reached or when the surface of the mixture softens or commences to melt. Because of the agglomeration that commences and the increase in friction associated therewith, the power increases that is taken up by the stirrer motor. As a rule, the granulation is terminated when the power uptake commences to rise exponentially. After that, the warm melt granulate is either discharged from the mixture and cooled in thin layers at room temperature or cooled with suitable cooling means (such as a cooling jacket) in the mixer, possibly with stirring. Pursuant to the invention, it is also possible to add the sucrose esters of the fatty acids in the molten state.

Surprisingly, a very narrow distribution of granulate sizes is achieved during this process. Depending on the manner, in which the process is carried out, the granulate or pellet particles have an almost rounded and smooth surface.

Likewise, it is possible to use other equipment, which can be heated, such as a fluidized bed granulator, or a rotor granulator.

The granules, so produced, can optionally be classified through a screen, possibly mixed with inert ingredients of the outer phase and, for example, pressed into tablets, or filled into capsules.

The customary pharmaceutical disintegrants or disintegrating agents, fillers, mold release agents or the like are used as inert materials of the outer phase. Usually, the use of mold release agents can be omitted when sucrose stearates of low HLB value are used, since sucrose stearates with a low HLB themselves also represent good mold release properties.

Accordingly, depending on the pharmaceutical, technological objective, rapidly releasing formulations and formulations, the release from which is modified to retarded (multiple units or single units), can be produced.

It was moreover surprisingly found that the sucrose esters of fatty acids are suitable as inert ingredients for hot melt coating. For this purpose, an amount of sucrose esters of fatty acids of the same or of a different type is added once again to a melt granulate, which has already been produced and solidified and the mixture is heated once again above the melting point or the softening temperature of the sucrose ester of the fatty acids added. The sucrose ester of the fatty acids is coated over the melt granulate at the same time. The coating process can also take place in the presence of a plasticizer. Likewise, granulates, which are free of sucrose esters of fatty acids or pure active ingredients can be coated in the manner described.

The advantage of this method is that, on the one hand, a sufficient control of the release, particularly a retardation, can be attained already by coating with smaller amounts of the sucrose esters of fatty acids. On the other hand, the surface of the granules or pellets, so prepared is smoothened.

A further advantage is that by this method coatings, which are resistant to gastric juices, can be produced in a simple manner. Thus, the possibility exists that the release of active ingredient in the acid range of the pH can be greatly retarded because the sucrose ester of fatty acids is practically insoluble in aqueous and acidic media.

Powder coating represents a special form of hot melt coating. On the one hand, the readily flowable sucrose esters of fatty acids are added with the help of a suitable powder feeder, and on the other hand, a plasticizer, such as triethyl citrate is added to the starting materials. This method is distinguished by large cost and time savings, since drying processes, such as those employed in conventional aqueous methods, are not required. In particular, the so prepared pharmaceutical formulations are suitable for water-sensitive active ingredients, such as Na valproate.

### BRIEF DESCRIPTION OF THE DRAWING

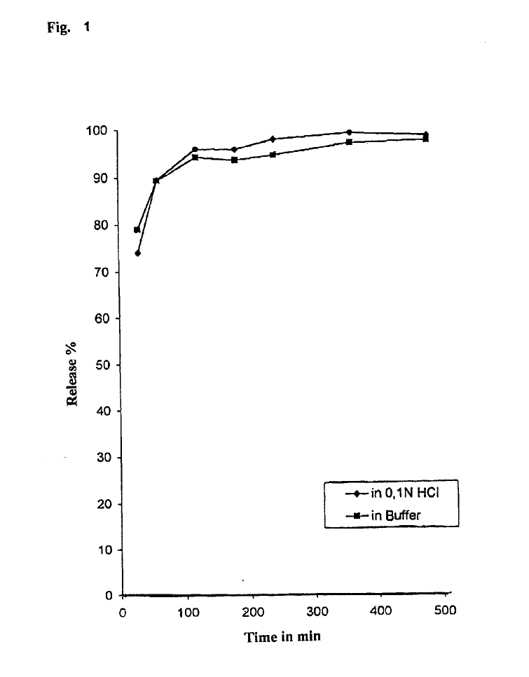

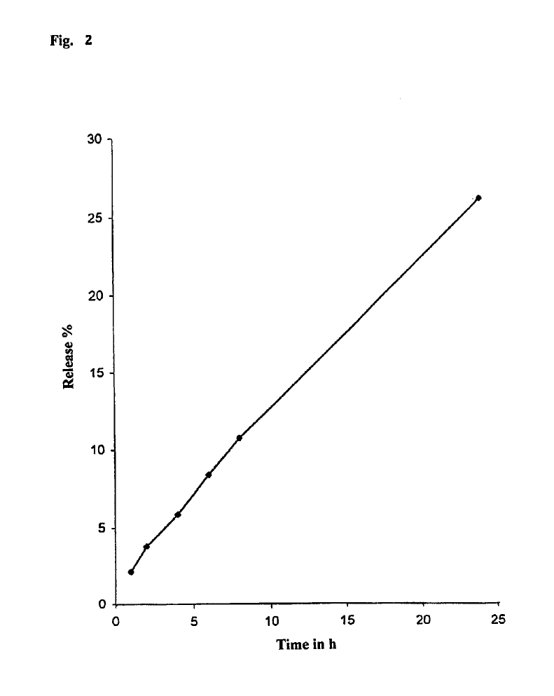

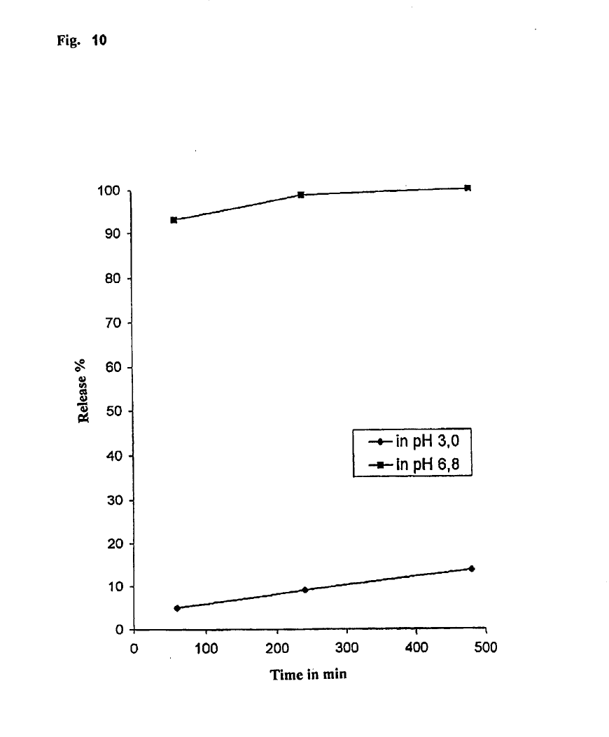

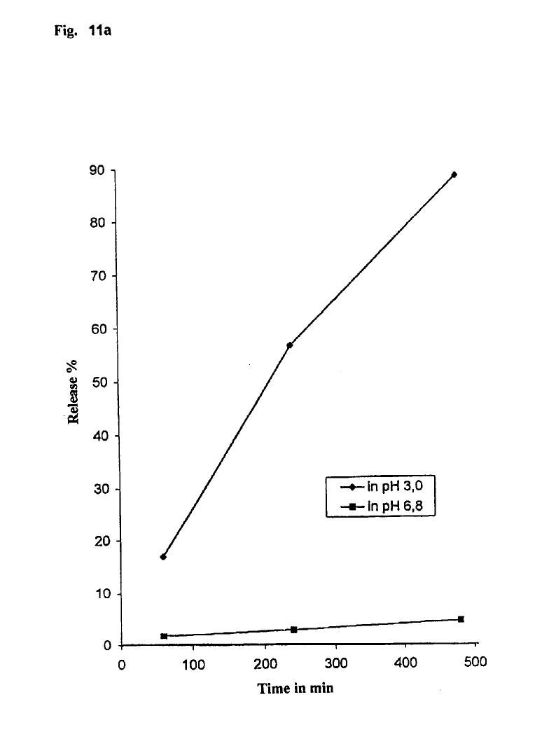

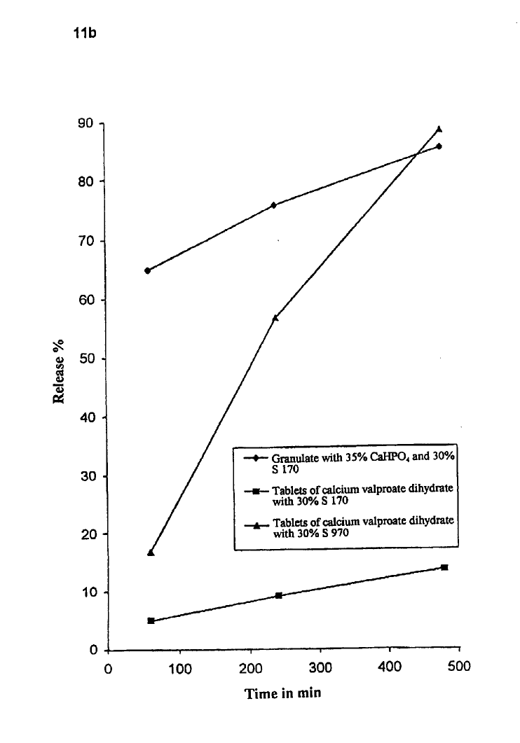

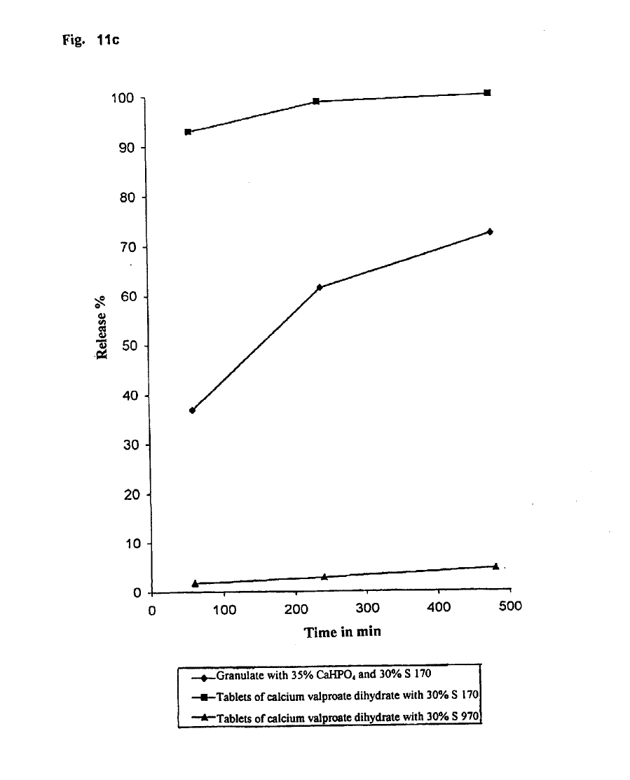

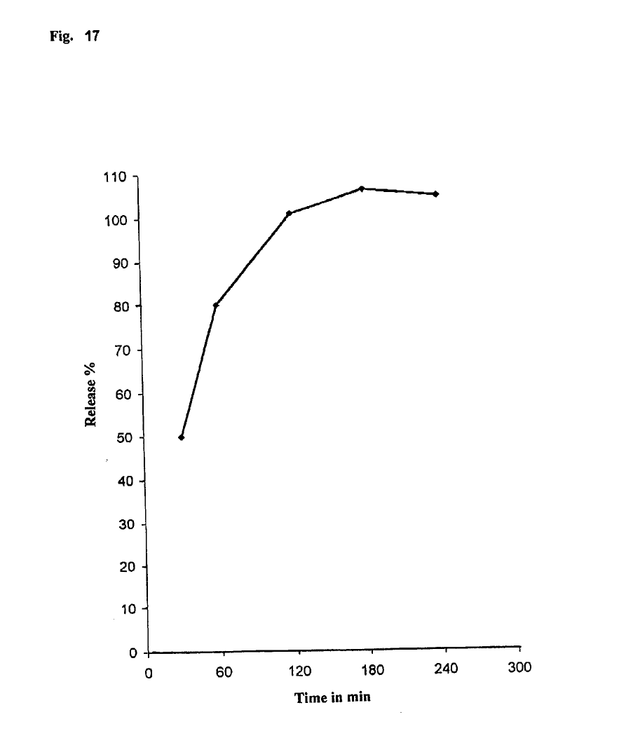

The invention is illustrated through the Examples and the appended drawing in which FIGS. 1-21 b illustrate properties of compositions as prepared by the Examples.

### DESCRIPTION OF PRFERRED EMBODIMENTS

The following examples further explain the present invention in greater detail.

### EXAMPLE 1

Tramadol Hydrochloride with 50% Sucrose Stearate with an HLB of 1

| | |
|---|---|
| Substance | Amount |

Formulation:

| | | |
|---|---|---|
| Tramadol hydrochloride | 400 | g |
| Sucrose stearate S-170 | 400 | g |

Parameters:

| | | |
|---|---|---|
| Amount formulated | 800 | g |
| impeller speed | 700 | rpm |
| Chopper speed | 3000 | rpm |
| Heater jacket temp. | 55.0° | C. |

The starting materials are heated with stirring in a high shear mixer of the GP1 type of firm Aeromatic-Fielder at the appropriate jacket temperature. The granulation commences when the product reaches a particular temperature. When the increase in the power uptake is reached and there is a sudden increase in the product temperature, the granulation is discontinued and the product is discharged, screened at a mesh width of 1.4 mm and cooled to room temperature.

Evaluation: Active Ingredient Release

| | Time in min | | | | | | |
|---|---|---|---|---|---|---|---|
| | 30 | 60 | 120 | 180 | 240 | 360 | 480 |
| Release in % | 74.03 | 89.40 | 95.75 | 95.57 | 97.61 | 98.58 | 97.87 |
| 0.1 N HC1 | | | | | | | |
| In buffer of pH 6.8 | 78.99 | 89.29 | 93.99 | 93.37 | 94.26 | 96.5 | 96.88 |

Active Ingredient Release: see FIG. 1

### EXAMPLE 2

Flupirtin maleate with 30% sucrose stearate of HLB 1

| | |
|---|---|
| Substance | Amount |

Formulation:

| | | |
|---|---|---|
| Flupirtin maleate | 240.0 | g |
| Sucrose stearate S-170 | 102.9 | g |

Parameters:

| | | |
|---|---|---|
| Amount of Formulation | 342.9 | g |
| Impeller speed | 700 | rpm |
| Chopper speed | 3000 | rpm |
| Heater jacket temp. | 61.2° | C. |

Produced as in Example 1

### EXAMPLE 3

Nifedipine with 30% sucrose stearate of HLB 1

| | |
|---|---|
| Substance | Amount |

Formulation:

| | | |
|---|---|---|
| Nifedipine | 560 | g |
| Sucrose stearate S-170 | 240 | g |

Parameters:

| | | |
|---|---|---|
| Amount of Formulation | 800 | g |
| Stirrer speed | 700 | rpm |
| Chopper speed | 3000 | rpm |
| Mantle temperature | 58° | C. |

Produced as in Example 1

Evaluation: Active Ingredient Release

| | Time in hours | | | | | |
|---|---|---|---|---|---|---|
| | 1 | 2 | 4 | 6 | 8 | 24 |
| Release in % in purified | 2.14 | 3.76 | 5.84 | 8.42 | 10.72 | 25.91 |
| water/1.25% SDS | | | | | | |

### EXAMPLE 4

Nifedipine with 30% sucrose palmitate of HLB 1

| | |
|---|---|
| Substance | Amount |

Formulation:

| | | |
|---|---|---|
| Nifedipine | 560 | g |
| Sucrose palmitate P-170 | 240 | g |

Parameters:

| | | |
|---|---|---|
| Amount of formulation | 800 | g |
| Impeller speed | 700 | rpm |
| Chopper speed | 3000 | rpm |
| Heater jacket temp. | 52° | C. |

Preparation as in Example 1

Evaluation: Active Ingredient Release

| | Time in hours | | | | | |
|---|---|---|---|---|---|---|
| | 1 | 2 | 4 | 6 | 8 | 24 |
| Release in % in purified | 4.08 | 7.32 | 11.5 | 16.65 | 21.71 | 49.04 |
| water/1.25% SDS | | | | | | |

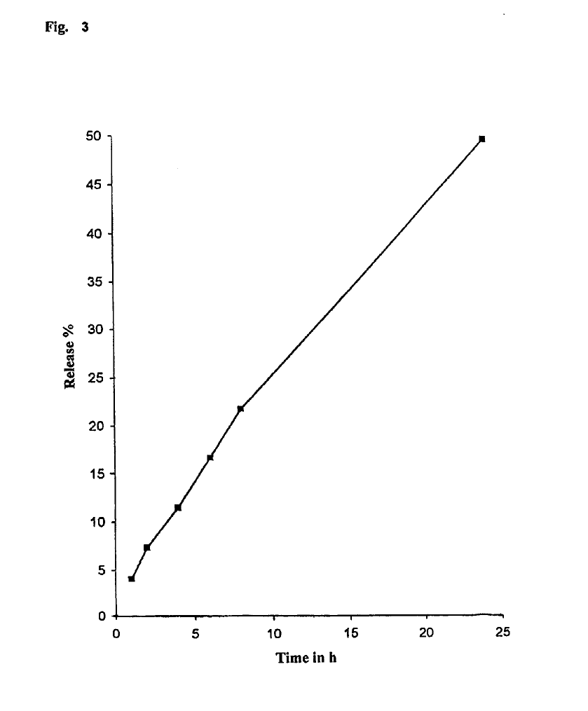

For Active Ingredient Release, see FIG. 3

### EXAMPLE 5

Tablets from melt granulate of nifedipine
and 30% sucrose stearate of HLB 5

| | |
|---|---|
| Substance | Amount |

Formulation:

| | | |
|---|---|---|
| Nifedipine | 560 | g |
| Sucrose stearate P-170 | 240 | g |

Parameters for granulating:

| | | |
|---|---|---|
| Amount of formulation | 800 | g |
| Impeller speed | 700 | rpm |
| Chopper speed | 3000 | rpm |
| Heater jacket temp. | 70° | C. |

Produced as in Example 1.

Parameters for tableting:

Subsequently, the granulate was pressed with a circular tableting tool with a diameter of 6 mm with medium convexity into tablets with a gross weight of 71.4 mg.

Evaluation: Active Ingredient Release

| | Time in min | | | | |
|---|---|---|---|---|---|
| | 30 | 60 | 120 | 180 | 240 |
| Release in % in purified | 19.85 | 42.44 | 78.30 | 96.61 | 102.88 |
| water/1.25% SDS | | | | | |

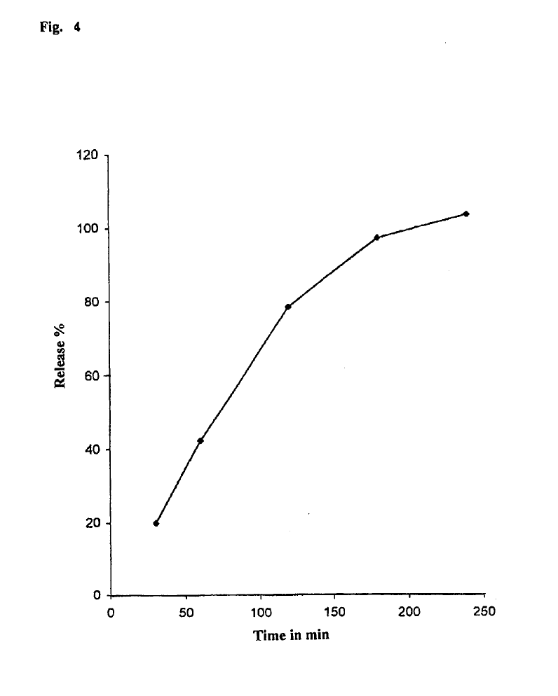

Active Ingredient Release: see FIG. 4

### EXAMPLE 6

Tablets from melt granulate of nifedipine with 50% sucrose
stearate of HLB 9 and 2.5% sucrose stearate of HLB 1

| | |
|---|---|
| Substance | Amount |

Formulation:

| | | |
|---|---|---|
| Nifedipine | 400 | g |
| Sucrose stearate S-970 | 380 | g |
| Sucrose stearate S-170 | 20 | g |

Parameters for granulating:

| | | |
|---|---|---|
| Amount of formulation | 800 | g |
| Impeller speed | 700 | rpm |
| Chopper speed | 3000 | rpm |
| Heater jacket temp. | 65° | C. |

Produced according to Example 1

Parameters for tableting:

Subsequently, the granulate was pressed with a circular tableting tool with a diameter of 6 mm with medium convexity into tablets with a gross weight of 100 mg.

Evaluation of Active Ingredients Release:

| | Time in min | | | | |
|---|---|---|---|---|---|
| | 30 | 60 | 120 | 180 | 240 |
| Release in % in purified | 20.10 | 40.37 | 73.26 | 94.14 | 102.93 |
| water/1.25% SDS | | | | | |

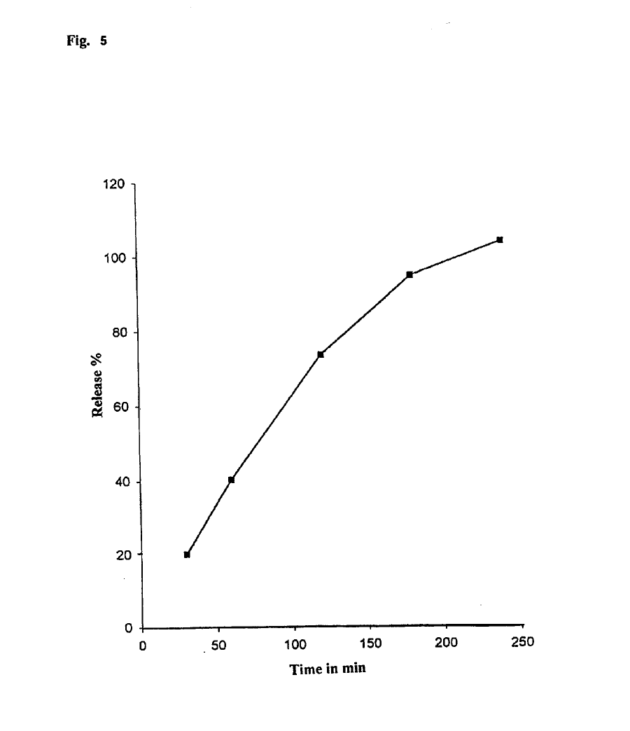

Active Ingredient Release: see FIG. 5

### EXAMPLE 7

Carbamazepine with 10% sucrose stearate of HLB 1

| | |
|---|---|
| Substance | Amount |

Formulation:

| | | |
|---|---|---|
| Carbamazepine | 720 | g |
| Sucrose stearate S-170 | 80 | g |

Parameters:

| | | |
|---|---|---|
| Amount of formulation | 800 | g |
| Impeller speed | 700 | rpm |
| Chopper speed | 3000 | rpm |
| Heater jacket temp. | 55° | C. |

Prepared according to Example 1

Evaluation: Active Ingredient Release

| | Time in min | | | | | | |
|---|---|---|---|---|---|---|---|
| | 30 | 60 | 120 | 180 | 240 | 360 | 480 |
| Release in % in | 10.68 | 20.06 | 38.08 | 51.45 | 62.47 | 73.89 | 81.58 |
| modified | | | | | | | |
| intestinal juice | | | | | | | |

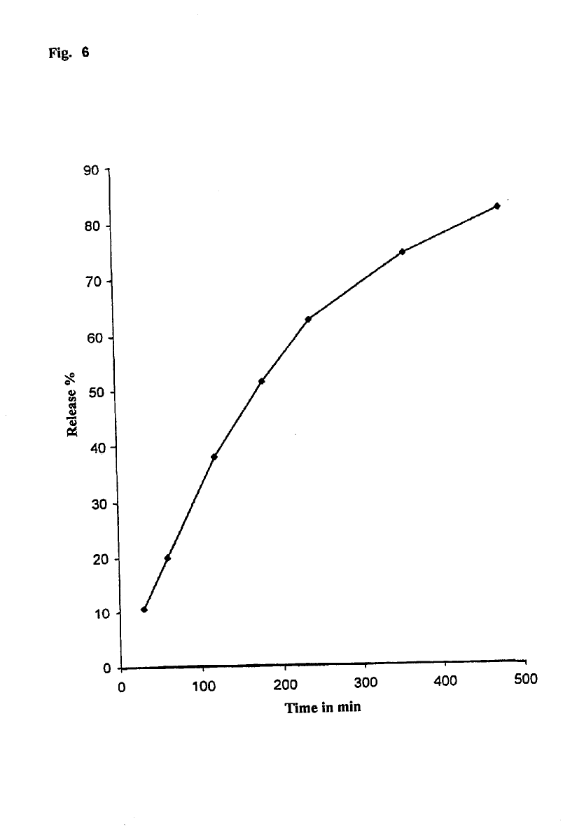

Active Ingredient Release: see FIG. 6

### EXAMPLE 8

Carbamazepine with 30% sucrose stearate of HLB 9

| | |
|---|---|
| Substance | Amount |

Formulation:

| | | |
|---|---|---|
| Carbamazepine | 560 | g |
| Sucrose stearate S-970 | 240 | g |

Parameters:

| | | |
|---|---|---|
| Amount of formulation | 800 | g |
| Impeller speed | 700 | rpm |
| Chopper speed | 3000 | rpm |
| Heater jacket temp. | 68° | C. |

Prepared according to Example 1

Evaluation: Active Ingredient Release

| | Time in min | | | | | | |
|---|---|---|---|---|---|---|---|
| | 30 | 60 | 120 | 180 | 240 | 360 | 480 |
| Released in % in | 26.09 | 42.27 | 62.65 | 80.58 | 87.38 | 96.56 | 100.84 |
| modified | | | | | | | |
| intestinal juice | | | | | | | |

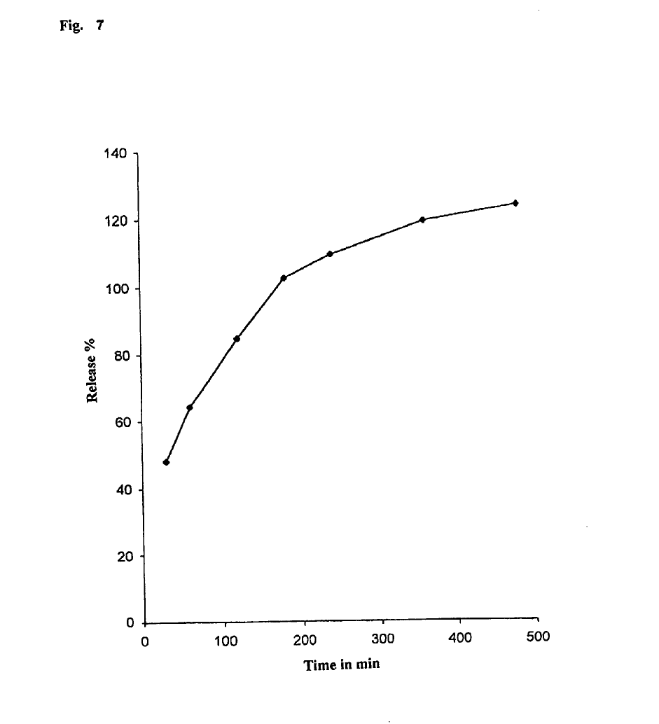

Active Ingredient Release: see FIG. 7

### EXAMPLE 9

Carbamazepine with 50% sucrose behenate
of HLB 3 and 2.5% triethyl citrate

| | |
|---|---|
| Substance | Amount |

Formulation:

| | | |
|---|---|---|
| Carbamazepine | 400 | g |
| Sucrose behenate B-370 | 380 | g |
| Triethyl citrate | 20 | g |

Parameters:

| | | |
|---|---|---|
| Amount of formulation | 800 | g |
| Impeller speed | 700 | rpm |
| Chopper speed | 3000 | rpm |
| Heater jacket temp. | 50° | C. |

The starting materials, carbamazepine and triethyl citrate, are mixed in anintensive mixer of the GP 1 type of firm Aeromatic-Fielder. After a mixing time of 1 minute, sucrose behenate B-3 70 is added and the mixture is heated with stirring at a heater jacket temperature of 50.0° C. When a particular product temperature is reached, at which an increase in power may be noted, the granulate is added to a screen with a mesh width of 1.4 mm and cooled to room temperature.

### EXAMPLE 10

Tablets of a melt granulate of carbamazepine with 30% sucrose
stearate of HLB 9

| | |
|---|---|
| Substance | Amount |

Formulation:

| | | |
|---|---|---|
| Carbamazepine | 560 | g |
| Sucrose stearate S-970 | 240 | g |

Parameters for granulating:

| | | |
|---|---|---|
| Amount of formulation | 800 | g |
| Impeller speed | 700 | rpm |
| Chopper speed | 3000 | rpm |
| Heater jacket temp. | 68° | C. |

Prepared according to Example 1

Parameters for tableting:

The granulate is pressed without further additions with a round tableting tool with a diameter of 13 mm into flat tablets with a gross weight of 571 mg and a crush strength of 25 N.

Evaluation: Release of Active Ingredient

| | Time in min | | | | | | |
|---|---|---|---|---|---|---|---|
| | 30 | 60 | 120 | 180 | 240 | 360 | 480 |
| Released in % in | 5.36 | 8.04 | 13.78 | 17.89 | 21.01 | 27.31 | 32.08 |
| modified intestinal | | | | | | | |
| juice | | | | | | | |

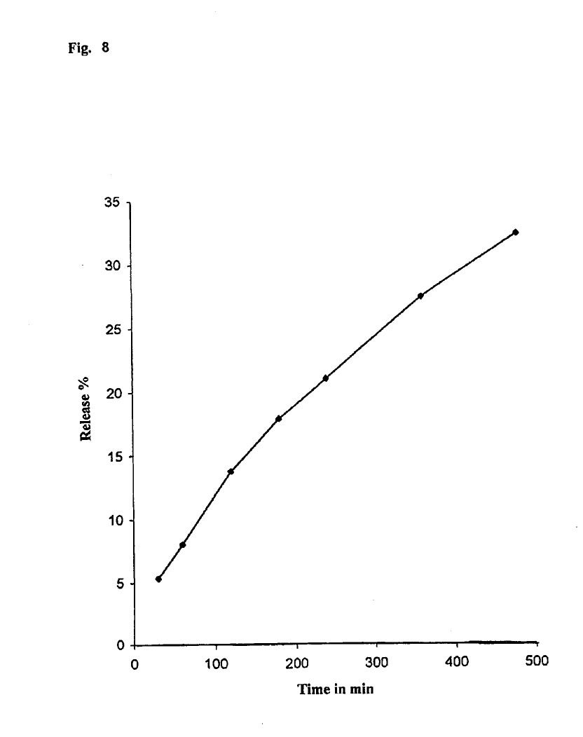

Active Ingredient Release: see FIG. 8

### EXAMPLE 11

Carbamazepine with 20% sucrose stearate of HLB 2

| | |
|---|---|
| Substance | Amount |

Formulation:

| | | |
|---|---|---|
| Carbamazepine | 640 | g |
| Sucrose stearate S-270 | 160 | g |

Parameters:

| | | |
|---|---|---|
| Amount of formulation | 800 | g |
| Impeller speed | 700 | rpm |
| Chopper speed | 3000 | rpm |
| Heater jacket temp. | 54° | C. |

Prepared as in Example 1

### EXAMPLE 12

Calcium valproate dihydrate with 35% calcium hydrogen phosphate
and 30% sucrose stearate of HLB 1

| | |
|---|---|
| Substance | Amount |

Formulation:

| | | |
|---|---|---|
| Calcium valproate dihydrate | 280 | g |
| Calcium hydrogen phosphate | 280 | g |
| Sucrose stearate S-170 | 240 | g |

Parameters:

| | | |
|---|---|---|
| Amount of formulation | 800 | g |
| Impeller speed | 700 | rpm |
| Chopper speed | 3000 | rpm |
| Heater jacket temp. | 53° | C. |

Preparation as in Example 1, the active ingredient calcium valproate dihydrate and calcium hydrogen phosphate being added to the mixer.

Evaluation: Active Ingredient Release

| | Time in min | | |
|---|---|---|---|
| | 60 | 240 | 480 |
| Released in % in pH 3.0 | 64.89 | 75.63 | 85.02 |
| Release in % in pH 6.8 | 36.85 | 61.26 | 71.60 |

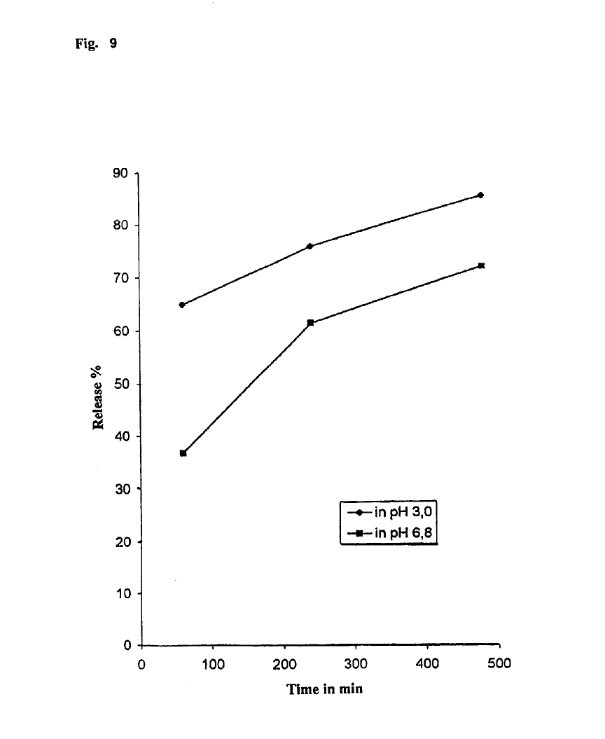

Active Ingredient Release: See FIG. 9

### EXAMPLE 13

Tablets from melt granulate of calcium valproate dihydrate and
30% sucrose stearate of HLB 1

| | |
|---|---|
| Substance | Amount |

Formulation:

| | | |
|---|---|---|
| Calcium valproate dihydrate | 560 | g |
| Sucrose stearate S-170 | 240 | g |

Parameters for granulating:

| | | |
|---|---|---|
| Amount of formulation | 800 | g |
| Impeller speed | 700 | rpm |
| Chopper speed | 3000 | rpm |
| Heater jacket temp. | 55° | C. |

Prepared according to Example 1

Parameters for tableting:

The granulate is pressed with an oblong tableting tool, 23 mm long, 9 mm wide, into oblong tablets with a gross weight of 951 mg and crush strength of 65 N.

Evaluation: Active Ingredient Release:

| | Time in min | | |
|---|---|---|---|
| | 60 | 240 | 480 |
| Released in % in pH 3.0 | 4.96 | 9.14 | 13.66 |
| Release in % in pH 6.8 | 92.93 | 98.57 | 99.43 |

Prepared according to Example 10

### EXAMPLE 14

Tablets from melt granulate of calcium valproate dihydrate
and 30% sucrose stearate of HLB 9

| | |
|---|---|
| Substance | Amount |

Formulation:

| | | |
|---|---|---|
| Calcium valproate dihydrate | 560 | g |
| Sucrose stearate S-970 | 240 | g |

Parameters for granulating:

| | | |
|---|---|---|
| Amount of formulation | 800 | g |
| Impeller speed | 700 | rpm |
| Chopper speed | 3000 | rpm |
| Heater jacket temp. | 65° | C. |

Preparation as in Example 1

Parameters for tableting:

The granulate is pressed with an oblong tableting tool, 23 mm long, 9 mm wide, into oblong tablets with a gross weight of 951 mg and crush strength of 50 N.

Evaluation: Active Ingredient Release

| | Time in min | | |
|---|---|---|---|
| | 60 | 240 | 480 |
| Released in % in pH 3.0 | 16.89 | 56.55 | 87.96 |
| Release in % in pH 6.8 | 1.75 | 2.77 | 4.34 |

Active Ingredient Release: see FIG. 11 a

For a comparison of the Active Ingredient Release of calcium valproate formulations at a pH of 3.0, see FIG. 11 b.

For a comparison of the Active Ingredient Release of calcium valproate formulations at a pH of 6.8, see FIG. 11 c.

### EXAMPLE 15

Retigabine with 20% sucrose stearate of HLB 1

| | |
|---|---|
| Substance | Amount |

Formulation:

| | | |
|---|---|---|
| Retigabine | 800 | g |
| Sucrose stearate S-170 | 200 | g |

Parameters:

| | | |
|---|---|---|
| Amount of formulation | 1000 | g |
| Impeller speed | 700 | rpm |
| Chopper speed | 3000 | rpm |
| Heater jacket temp. | 52° | C. |

Prepared according to Example 1

Evaluation: Active Ingredient Release

| | Time in min | | | | | | |
|---|---|---|---|---|---|---|---|
| | 30 | 60 | 120 | 180 | 240 | 360 | 480 |
| Release in | 37.23 | 46.71 | 75.81 | | | | |
| % in 0.1 N HC1 | | | | | | | |
| In a buffer at a pH of | 5.71 | 8.77 | 13.82 | 15.79 | 23.60 | 27.99 | 35.62 |
| 6-8/1% Texapon | | | | | | | |

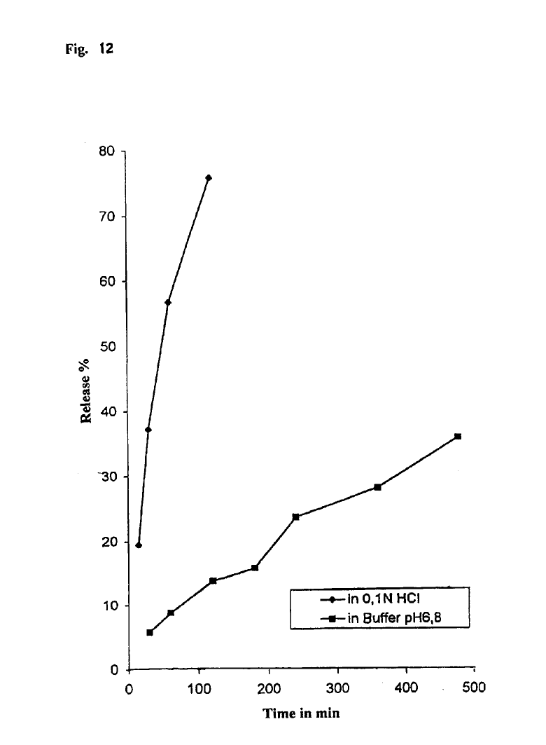

Active Ingredient Release: see FIG. 12

### EXAMPLE 16

Retigabine with 20% sucrose stearate of HLB 2

| | |
|---|---|
| Substance | Amount |

Formulation:

| | | |
|---|---|---|
| Retigabine | 400 | g |
| Sucrose stearate S-270 | 100 | g |

Parameters:

| | | |
|---|---|---|
| Amount of formulation | 500 | g |
| Impeller speed | 700 | rpm |
| Chopper speed | 3000 | rpm |
| Heater jacket temp. | 55° | C. |

Prepared according to Example 1

Evaluation: Active Ingredient Release

| | Time in min | | | | | | | |
|---|---|---|---|---|---|---|---|---|
| | 15 | 30 | 60 | 120 | 180 | 240 | 360 | 480 |
| Release | 42.28 | 62.58 | 83.53 | 100.97 | | | | |
| in % in | | | | | | | | |
| 0.1 N HC1 | | | | | | | | |
| Release | | 11.82 | 20.77 | 34.41 | 44.94 | 52.74 | 61.63 | 66.37 |
| in % in | | | | | | | | |
| buffer of | | | | | | | | |
| pH 7.5 | | | | | | | | |

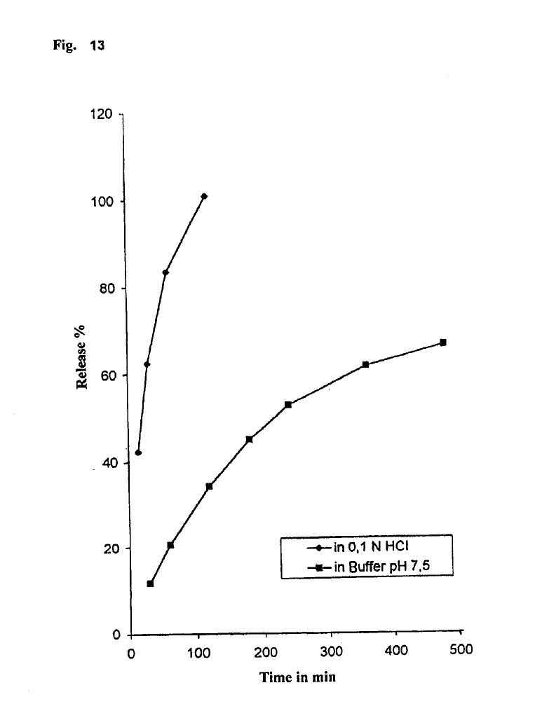

Active Ingredient Release: see FIG. 13

### EXAMPLE 17

Retigabine with 20% sucrose stearate of HLB 1 and
10% sucrose stearate of HLB 9

| | |
|---|---|
| Substance | Amount |

Formulation:

| | | |
|---|---|---|
| Retigabine | 210 | g |
| Sucrose stearate S-170 | 60 | g |
| Sucrose stearate S-970 | 30 | g |

Parameters:

| | | |
|---|---|---|
| Amount of formulation | 300 | g |
| Impeller speed | 500 | rpm |
| Chopper speed | 3000 | rpm |
| Heater jacket temp. | 65° | C. |

Preparation as in Example 1

Evaluation: Active Ingredient Release:

| | Time in min | | | | | | | |
|---|---|---|---|---|---|---|---|---|
| | 15 | 30 | 60 | 120 | 180 | 240 | 360 | 480 |
| Release in | 71.49 | 85.13 | 97.93 | 102.82 | | | | |
| % in | | | | | | | | |
| 0.1 N HCl | | | | | | | | |
| Release in % | | 31.02 | 36.93 | 51.97 | 61.25 | 70.63 | 79.01 | 78.77 |
| in a | | | | | | | | |
| buffer of | | | | | | | | |
| pH 6.8 | | | | | | | | |

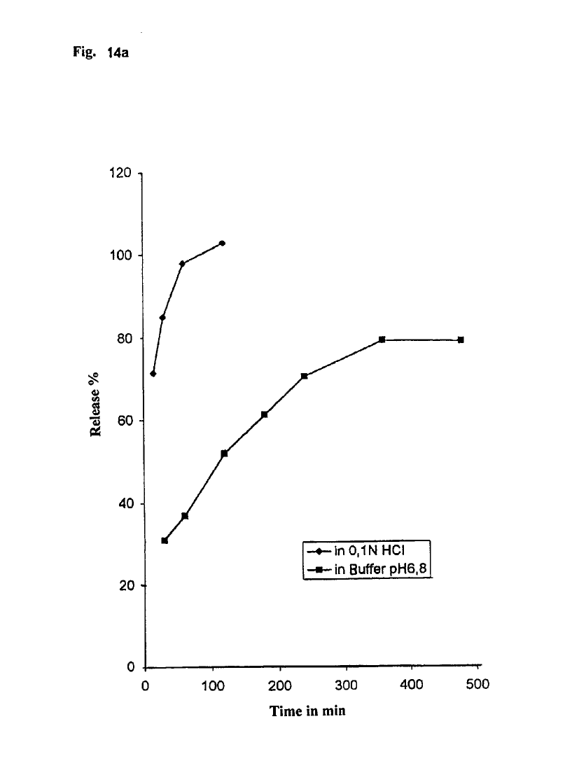

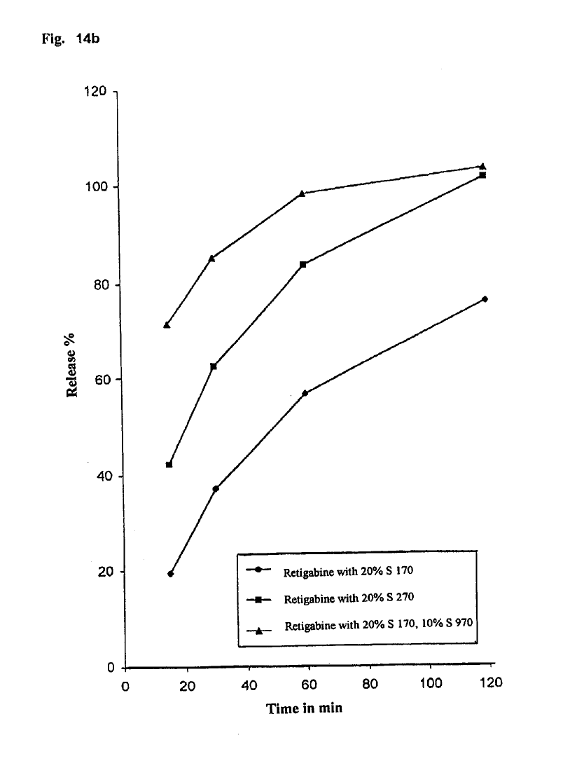

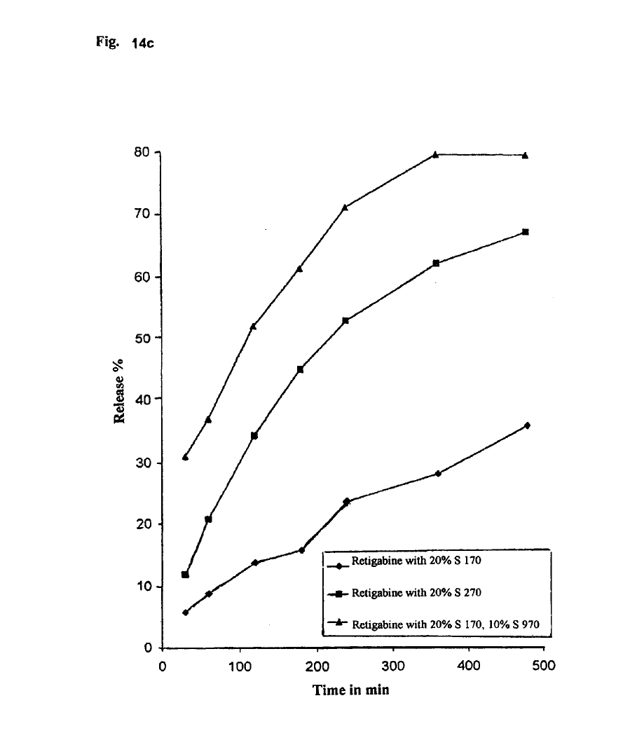

Active Ingredient Release: see FIG. 14 a.

For a comparison of the Active Ingredient Release from Retigabine formulations in 0.1 N HC1, see FIG. 14 b.

For a comparison of the Active Ingredient Release from Retigabine formulations in a buffer of pH 6.8, see FIG. 14 c

### EXAMPLE 18

Tablets of melt granulate with retigabine, 20% sucrose stearate
of HLB 1 and 10% croscarmelose sodium

| | |
|---|---|
| Substance | Amount |

Formulation:

| | | |
|---|---|---|
| Retigabine | 800 | g |
| Sucrose stearate S-170 | 200 | g |

Parameters for granulating:

| | | |
|---|---|---|
| Amount of formulation | 1000 | g |
| Impeller speed | 700 | rpm |
| Chopper speed | 3000 | rpm |
| Heater jacket temp. | 52° | C. |

Prepared according to Example 1

Parameters for tableting:

| Substance | Amount |
|---|---|
| Retigabine retard - granulate (see above) | 270 g |
| Croscarmellose sodium | 30 g |

The tableting mixture is pressed into tablets with a round tableting tool with a diameter of 9 mm, a bevel of 45°, and a radius of curvature R13.

Evaluation: Active Ingredient Release:

| | Time in min | | | | | | | |
|---|---|---|---|---|---|---|---|---|
| | 15 | 30 | 60 | 120 | 180 | 240 | 360 | 480 |
| Release in | 40.76 | 81.59 | 96.13 | 100.76 | | | | |
| % in 0.1 N | | | | | | | | |
| HC1 | | | | | | | | |
| In a buffer | | 22.20 | 29.80 | 38.95 | 46.49 | 53.58 | 60.85 | 64.69 |
| of pH | | | | | | | | |
| 6.8/1% | | | | | | | | |
| sodium | | | | | | | | |
| dodecyl | | | | | | | | |
| sulfate | | | | | | | | |
| (SDS) | | | | | | | | |

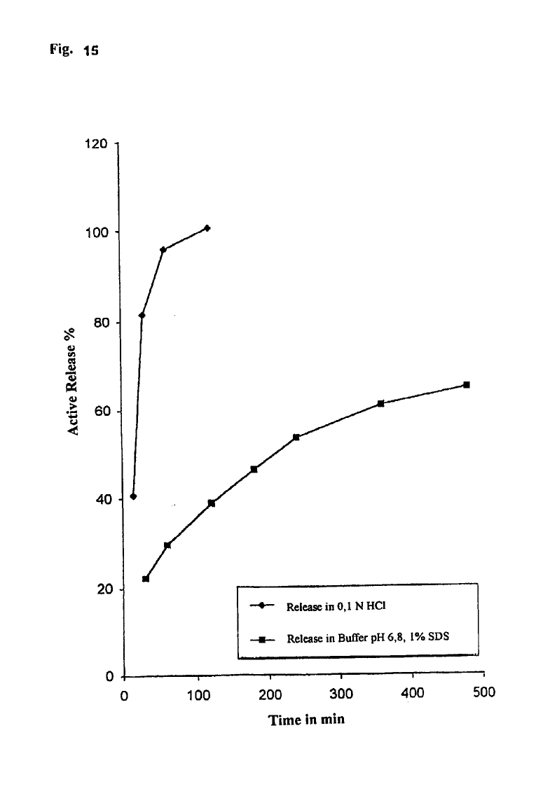

Active Ingredient Release: see FIG. 15

### EXAMPLE 19

Retigabine with 7% sucrose stearate of HLB 1

| | |
|---|---|
| Substance | Amount |

Formulation:

| | | |
|---|---|---|
| Retigabine | 372 | g |
| Sucrose stearate S-170 | 28 | g |

Parameters:

| | | |
|---|---|---|
| Amount of formulation | 400 | g |
| Impeller speed | 1300 | rpm |
| Heater jacket temp. | 50° | C. |

In a high shear mixer of the GP 1 type of firm Aeromatic-Fielder, the starting materials are heated with stirring at a mantle temperature of 50.0° C. in a special bowl, which is provided with a PTFE in-liner. After the power consumption uptake has increased once again, the pellets are removed and cooled to room temperature in thin layers.

Evaluation: Active Ingredient Release:

| | Time in min | | | | | | |
|---|---|---|---|---|---|---|---|
| | 30 | 60 | 120 | 180 | 240 | 360 | 480 |
| Release in a buffer of | 16.62 | 29.85 | 50.39 | 67.14 | 69.35 | 83.20 | 90.96 |
| pH 7.5/2.5 SDS | | | | | | | |

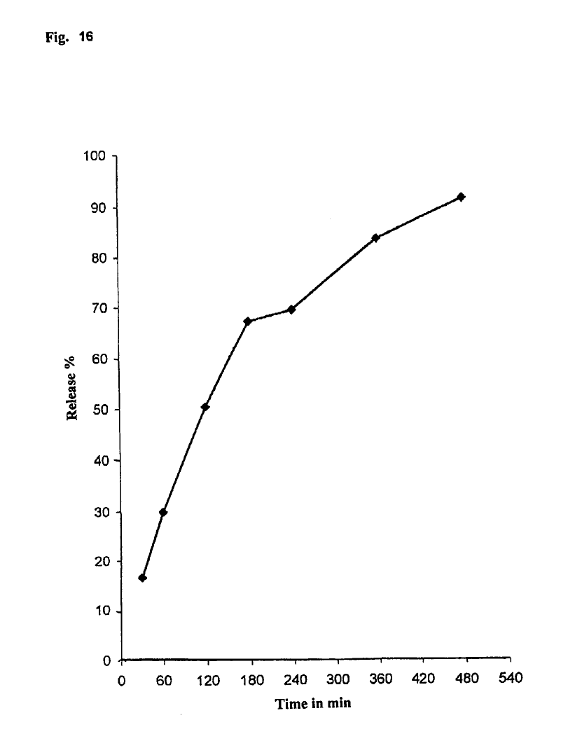

Active Ingredient Release: see FIG. 16

### EXAMPLE 20

Retigabine with 20% sucrose stearate of HLB 11

| | |
|---|---|
| Substance | Amount |

Formulation:

| | | |
|---|---|---|
| Retigabine | 320 | g |
| Sucrose stearate S-1170 | 80 | g |

Parameters:

| | | |
|---|---|---|
| Amount of formulation | 400 | g |
| Impeller speed | 1300-1100 | rpm |
| Heater jacket temp. | 50° | C. |

Prepared according to Example 19

Evaluation: Active Ingredient Release

| | Time in min | | | | |
|---|---|---|---|---|---|
| | 30 | 60 | 120 | 180 | 240 |
| Release in a buffer of | 49.91 | 79.95 | 100.81 | 106.03 | 104.36 |
| pH 7.5/2.5% SDS | | | | | |

Active Ingredient Release: see Example 17

### EXAMPLE 21

Retigabine with 20% sucrose stearate of HLB 16

| | |
|---|---|
| Substance | Amount |

Formulation:

| | | |
|---|---|---|
| Retigabine | 320 | g |
| Sucrose stearate S-1170 | 80 | g |

Parameters for
granulating:

| | | |
|---|---|---|
| Amount of formulation | 400 | g |
| Impeller speed | 1300-1100 | rpm |
| Heater jacket temp. | 50-55° | C. |

Prepared according to Example 19

Evaluation: Active Ingredient Release

| | Time in min | | | | |
|---|---|---|---|---|---|
| | 30 | 50 | 120 | 180 | 240 |
| Released in a buffer of | 41.77 | 68.71 | 92.32 | 99.95 | 101.47 |
| pH 7.5/2.5% SDS | | | | | |

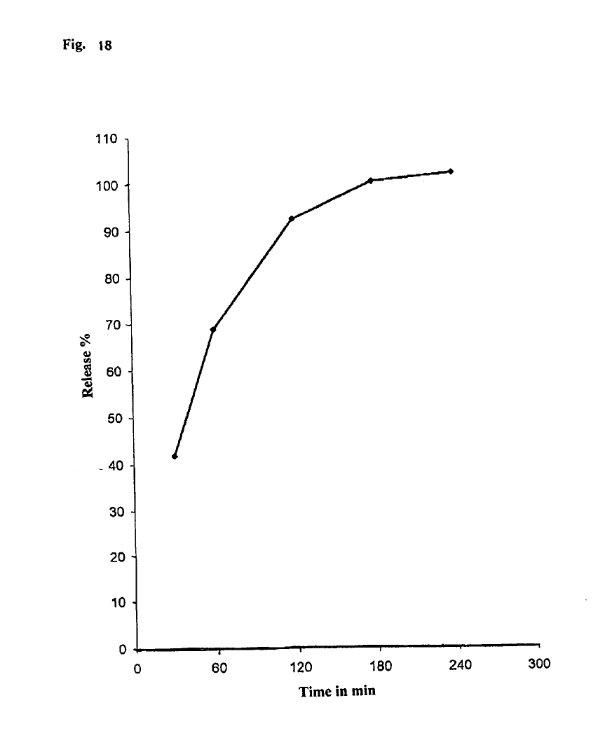

Active Ingredient Release: see FIG. 18

### EXAMPLE 22

Retigabine with 16% sucrose stearate of HLB 15

| | |
|---|---|
| Substance | Amount |

Formulation:

| | | |
|---|---|---|
| Retigabine | 336 | g |
| Sucrose stearate S-1570 | 64 | g |

Parameters:

| | | |
|---|---|---|
| Amount of formulation | 400 | g |
| Impeller speed | 1300 | rpm |
| Heater jacket temp. | 50-60° | C. |

Prepared according to Example 19

Evaluation: Active Ingredient Release

| | Time in min | | | | |
|---|---|---|---|---|---|
| | 30 | 60 | 120 | 180 | 240 |
| Release in a buffer of | 64.67 | 89.83 | 99.98 | 101.78 | 100.99 |
| pH 7.5/2.5% SDS | | | | | |

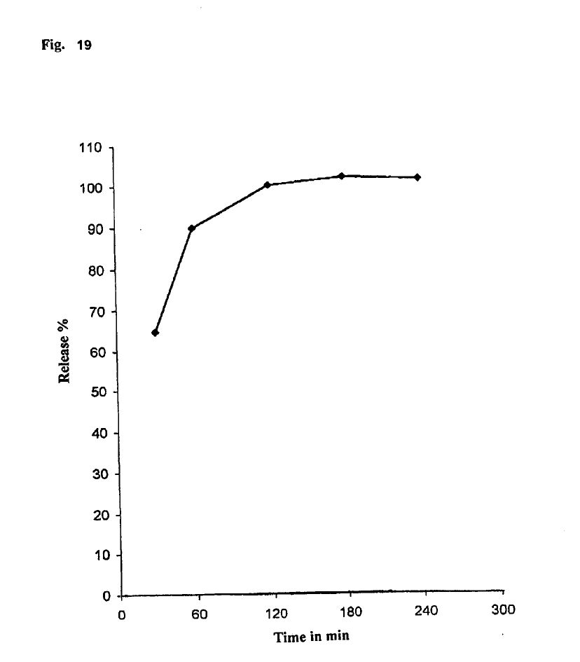

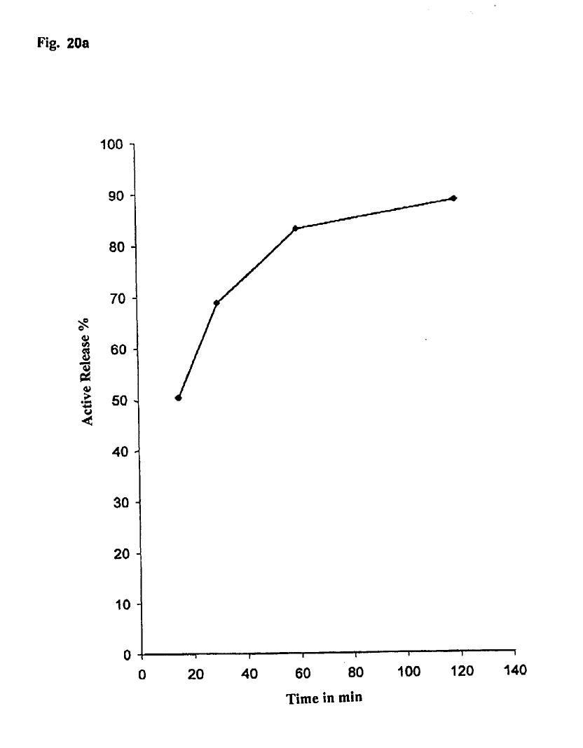

Active Ingredient Release: see FIG. 19

### EXAMPLE 23

Retigabine tablets

| | |
|---|---|
| Substance | Amount |

Formulation melt granulate

| | | |
|---|---|---|
| Retigabine | 332 | g |
| Sucrose stearate S-1570 | 68 | g |

Parameters:

| | | |
|---|---|---|
| Amount of formulation | 400 | g |
| Impeller speed | 1300 | rpm |
| Heater jacket temp. | 50-50° | C. |

Prepared according to Example 19

Coating formulation

| Substance | Amount |
|---|---|
| Retigabine - melt granulate with 17% | 336 g |
| sucrose stearate of HLB 15 | |
| Eudragit L 30 D-55 | 400 g |
| | (corresponds |
| | to 120 g of |
| | lacquer |
| | solids) |
| Talcum | 60 g |
| Triethyl citrate | 12 g |

The melt granulates from 5 batches are combined and sprayed in a rotor granulator with an inflow of air at 50° C. at 300 rpm with a suspension of Eudragit L 30 D-55, talcum and triethyl citrate in 536 g of purified water. This is followed by drying up to a product temperature of 33° C.

The granulate, so coated, is homogenized for 10 minutes in a Turbula with 30% by weight of microcrystalline cellulose and 5% by weight of croscarmellose sodium.

The tableting mixture is pressed into oblong 17×8 mm, curved tablets with an average crush strength of 87 N.

| | Time in min | | | | | |
|---|---|---|---|---|---|---|
| | 15 | 30 | 60 | 120 | 180 | 240 |
| Released in % in | 50.3 | 68.8 | 83 | 83.3 | | |
| 0.1N HC1 | | | | | | |
| In a buffer of pH 7.5/1.7% | 16.1 | 30.4 | 55.7 | 83.4 | 95.2 | 99.2 |
| SDS | | | | | | |

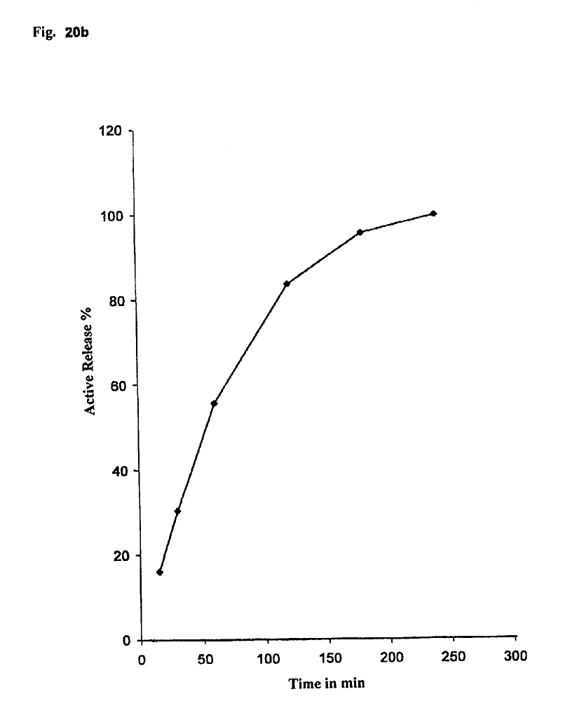

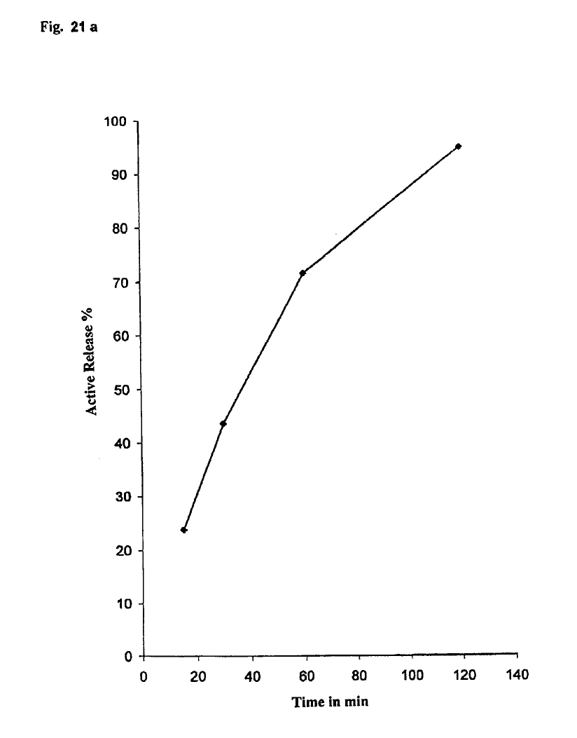

Active Ingredient Release in 0.1 N HC1: see FIG. 20 a

Active Ingredient Release in a buffer of 7.5, 1.7% SDS: see FIG. 20 b

### EXAMPLE 24

Hot melt coating of retigabine melt granulate with
10% sucrose stearate of HLB 1

| | |
|---|---|
| Substance | Amount |

Formulation of coated melt granulate:

| | | |
|---|---|---|
| Retigabine melt granulate (90% by weight of retigabine | 500 | g |
| and 10% by weight of sucrose Stearate S-170) | | |
| Sucrose stearate S-170 | 55.6 | g |

Parameters:

| | | |
|---|---|---|
| Amount of formulation | 555.6 | g |
| Impeller speed | 700 | rpm |
| Heater jacket temp. | 52° | C. |

In a high shear mixer of the GP 1 type of the firm Aeromatic-Fielder, the retigabine melt granulate is heated with stirring at a heater jacket temperature of 52° C. At a product temperature of 30° C., sucrose stearate S-170 is added and granulated for a further 7 minutes with the chopper switched on (3000 rpm). The coated granulate was removed and screened through a 1.4 mm mesh screen.

Results: particle size distribution

| Particle size (u.m.) | Percentage (%) |
|---|---|
| >1000 | 3.4 |
| >800 | 4.0 |
| >500 | 34.4 |
| >315 | 40.9 |
| >160 | 14.0 |
| >50 | 3.4 |
| >50 | 0 |

| | Time in min | | | | | | | |
|---|---|---|---|---|---|---|---|---|
| | 15 | 30 | 60 | 120 | 180 | 240 | 360 | 480 |
| Released | 23.8 | 43.6 | 71.4 | 94.4 | | | | |
| in % in | | | | | | | | |
| 0.1 N HC1 | | | | | | | | |
| In a buffer | | 7.2 | 13.2 | 19.8 | 25.4 | 31 | 40.2 | 47.4 |
| of pH | | | | | | | | |
| 7.5/2.5% SDS | | | | | | | | |

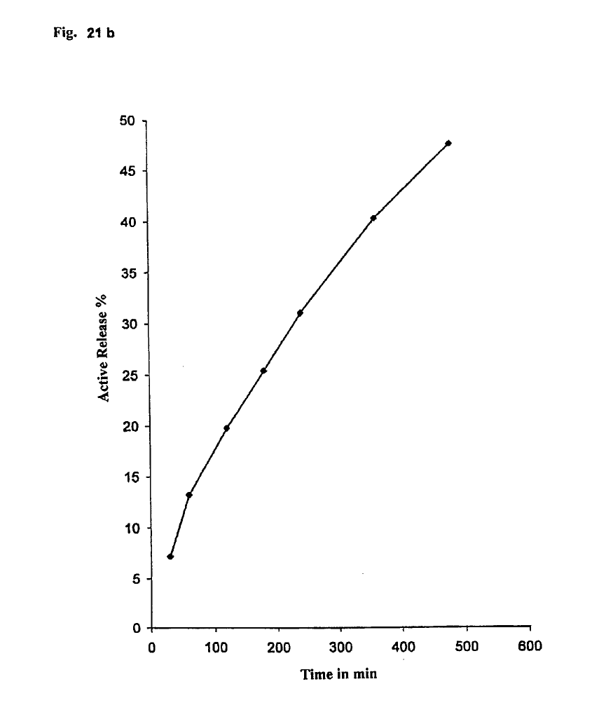

Active Ingredient Release in 0.1 N HC1: see FIG. 21 a.

Active Ingredient Release in a buffer of pH 7.5, 2.5% SDS: see FIG. 21 b.

What is claimed is:

1. An oral pharmaceutical formulation having variably adjustable release behavior, the oral pharmaceutical formulation comprising a granulated proportion as an inner phase, wherein the inner phase comprises at least one active ingredient and at least one fatty acid sucrose ester as the single release-controlling agent and wherein the inner phase is made by melt granulation or melt pelletization of the at least one active ingredient and the at least one fatty acid sucrose ester, wherein the at least one active ingredient and the at least one fatty acid sucrose aster are heated while being agitated to a temperature at which the at least one fatty acid sucrose ester softens at the surface or commences to melt or the melting temperature of the at least one fatty acid sucrose ester is reached so that agglomeration and granulation occur and granules are formed and, after termination of granulation, the granules are cooled.

2. The pharmaceutical formulation according to claim 1 as a single unit drug form or multiple unit drug form.

3. The pharmaceutical formulation according to claim 1 selected from the group of granulates, pellets, tablets, film coated tablets, microtablets, sugar coated tablets, and capsules.

4. The pharmaceutical formulation according to claim 1, wherein the at least one fatty acid sucrose ester is comprised of mono-, di-, tri- and polyesters of sucrose and saturated or unsaturated fatty acids.

5. The pharmaceutical formulation according to claim 4, wherein the at least one fatty acid sucrose ester comprises of C₁₂ to C₂₂ fatty acids.

6. The pharmaceutical formulation according to claim 1, wherein the HLB value of the at least one fatty acid sucrose ester is 1 to 16.

7. The pharmaceutical formulation according to claim 1, wherein the at least one fatty acid sucrose ester has a melting point or melting range in a temperature range of 30° C. to 200° C.

8. The pharmaceutical formulation according to claim 1, wherein the at least one fatty acid sucrose ester has a melting point or a melting range in a temperature range of 40° C. to 150° C.

9. The pharmaceutical formulation according to claim 1, comprising a coating, wherein the coating comprises at least one fatty acid sucrose ester, wherein the at least one fatty acid sucrose ester of the coating is present in an amount of 1% to 60% by weight based on the coated pharmaceutical formulation.

10. The pharmaceutical formulation according to claim 9, wherein the at least one fatty acid sucrose ester of the coating is present in an amount of 1% to 60% by weight based on the coated pharmaceutical formulation.

11. The pharmaceutical formulation according to claim 1, wherein the at least one active ingredient is selected from the group consisting of analeptic agents, antihypoxemic agents, analgesics, antirheumatic agents, antiallergic agents, antiarrhythmic agents, antidementia agents, antidiabetic agents, antiemetic agents, antivertiginous agents, antiepilieptic agents, antihypertensive agents, antihypotensive agents, broncholytic agents, antiasthmatic agents, diuretics, circulation promoters, hypnotic agents, sedatives, cardiac agents, lipid-lowering agents, antimigraine preparations, muscle relaxants, anti-Parkinson agents, and psycho-pharmaceuticals.

12. The pharmaceutical formulation according to claim 1, wherein the at least one active ingredient is selected from the group consisting of caffeine, diclofenac, morphine, tilidine, pentifylline, vincamine, azelastine, pseudoephedrine, quinidine, diltiazem, verapamil, piracetam, nicergoline, xantino nicotinate, glibenclamide, betahistin dimesilate, dimenhydrinate, valproic acid, talinolol, fosinopril, doxazosin, metoprolol, norfenefrine-HCl, dihydroergotamine mesilate, salbutamol, terbutaline sulfate, theophylline, furosemide, piretamide, buflomedil, naftidrofuryl, pentoxifylline, trinitroglycerin, isosorbide mononitrate, isosorbide dinitrate, molsidomine, bezafibrate, fenofibrate, xantinol, sumatriptan, levodopa, benserazide, carbidopa, amitriptyline HCl, venlafaxine-HCl, lithium carbonate, and lithium acetate.

13. The pharmaceutical formulation according to claim 1, wherein the at least one active ingredient is flupirtine, tramadol, nifedipine, carbamazepine, calcium valproate or retigabine.

14. A method for preparing an oral pharmaceutical formulation by melt granulation or melt pelletization, the method comprising the steps of:
heating at least one active ingredient and at least one fatty acid sucrose ester while being agitated to a temperature at which the at least one fatty acid sucrose ester softens at the surface or commences to melt or the melting temperature of the at least one fatty acid sucrose ester is reached so that agglomeration occurs and granules are formed; and
cooling the granules after termination of granulation.

15. The method according to claim 14, wherein in the step of heating the at least one fatty acid sucrose ester is heated and melted separately and added in melted form to the at least one active ingredient.

16. The method according to claim 14, wherein the step of heating is carried out in a high speed mixer, a high shear mixer, a fluidized bed, or a rotor granulator to provide agitation.

17. The method according to claim 14, further comprising the step of coating the granules by hot melt coating or by powder coating.

18. The method according to claim 17, wherein in the step of coating at least one fatty acid sucrose ester is employed alone or in combination with at least one plasticizer for producing the coating.

19. The method according to claim 18, wherein the at least one plasticizer is selected from the group consisting of triethyl citrate, acetyl triethyl citrate, triacetin and dibutyl sebacate.
